(19) United States
(12) Patent Application Publication
(10) Pub. No.: US 2022/0180168 A1
(43) Pub. Date: Jun. 9, 2022

(54) INTEGRATED SEGMENTATION AND INTERPRETABLE PRESCRIPTIVE POLICIES GENERATION

(71) Applicant: International Business Machines Corporation, Armonk, NY (US)

(72) Inventors: Max Biggs, Charlottesville, VA (US); Wei Sun, Tanytown, NY (US); Shivaram Subramanian, Frisco, TX (US); Markus Ettl, Yorktown Heights, NY (US)

(21) Appl. No.: 17/111,212

(22) Filed: Dec. 3, 2020

(51) Int. Cl. G06N 3/08; G06N 5/00; G06N 3/04; G06K 9/62; G16H 20/00; G06Q 30/02

(52) U.S. Cl. CPC G06N 3/08; G06N 5/003; G06Q 30/0211; G06K 9/6256; G16H 20/00; G06N 3/04

(57) ABSTRACT

One embodiment of the invention provides a method for integrated segmentation and prescriptive policies generation. The method comprises training a first artificial intelligence (AI) model and a second model based on training data. The first AI model comprises a teacher model trained to determine a likelihood of a desired outcome for a given action. The second model comprises a prescriptive tree trained for segmentation. The method further comprises determining, via the teacher model, a first policy that produces an optimal action. The optimal action provides a best expected outcome. The method further comprises applying, via the second model, a recursive segmentation algorithm to generate one or more interpretable prescriptive policies. Each interpretable prescriptive policy is less complex and more interpretable than the first policy. The method further comprises, for each interpretable prescriptive policy, determining, via the teacher model, an expected outcome for the interpretable prescriptive policy.

### BACKGROUND

The field of embodiments of the invention generally relate to artificial intelligence (AI).

Despite recent surge of interest in making prediction models more interpretable (i.e., reasoning), comparatively there is significantly less work on interpreting policies from these models when embedded in an operational decision-making context. A successful predictive model does not result in a successful prescriptive model. For example, if a tree-based predictive model includes a partition of data which leads to successful predictive accuracy (e.g., predicting purchase probability), the same model does not necessarily translate to a successful prescriptive decision (e.g., revenue-maximizing prices).

Conventional solutions implement an as-is process for generating interpretable policies that involves segmentation followed by optimization. One example conventional solution for an application use involving pricing builds segments by training a decision tree to classify data into different groups based on purchase information indicative of a population's propensity to purchase, where each path of the decision tree represents a segment of the population. Another example conventional solution for an application use involving pricing builds segments by utilizing an unsupervised clustering technique (e.g., K-means) to obtain clusters/segments, without using purchase information. Each segment of the population (obtained via the decision tree or the clustering technique) is assumed to be homogeneous in terms of willingness to pay and sensitivity to price. The number of segments/rules is typically determined in an ad-hoc fashion. As these conventional solutions generate segments without considering revenue maximization, customers in the same segment share similar propensity to purchase, there could be significant heterogeneity in price responses among customers in the same segment even if the customers have similar propensity to purchase.

Further, to implement price/policy optimization, these conventional solutions train a demand model or each segment based on price information (optionally with other features), and determine an optimal price that maximizes expected revenue. One key limitation of this approach is that a segment defined is to minimize classification error, not maximize revenue. Another key limitation of this approach is a restrictive assumption which requires homogeneity of price elasticity within each segment.

Complex and opaque AI prediction models (e.g., boosted trees, neural networks) make it difficult for decision-makers to understand and trust them, resulting in a reluctance in AI adoption in practice despite their potential benefits. There is need to produce accurate and interpretable prescriptive decisions. For example, for an application use involving pricing, having a limited number of price rules is preferable. There is a need to quantify trade-off between accuracy and interpretability to provide guidance to a decision-maker. If the cost of interpretability (i.e., difference in outcome between a complex policy and a simple policy) is significant, a higher cost of implementation for interpretability is justified.

### SUMMARY

Embodiments of the invention generally relate to artificial intelligence (AI), and more specifically, to a method and system for integrated segmentation and prescriptive policies generation.

One embodiment of the invention provides a method for integrated segmentation and prescriptive policies generation. The method comprises training a first AI model and a second model based on training data. The first AI model comprises a teacher model trained to determine a likelihood of a desired outcome for a given action. The second model comprises a prescriptive tree trained for segmentation. The method further comprises determining, via the teacher model, a first policy that produces an optimal action. The optimal action provides a best expected outcome. The method further comprises applying, via the second model, a recursive segmentation algorithm to generate one or more interpretable prescriptive policies. Each interpretable prescriptive policy is less complex and more interpretable than the first policy, from the teacher model, that produces the optimal action. The method further comprises, for each interpretable prescriptive policy, determining, via the teacher model, an expected outcome for the interpretable prescriptive policy. Other embodiments include a system for integrated segmentation and prescriptive policies generation, and a computer program product for integrated segmentation and prescriptive policies generation. These features contribute to the advantage of providing accurate and interpretable prescriptive decisions.

One or more of the following features may be included. In some embodiments, for each interpretable prescriptive policy, a difference between the best expected outcome and an expected outcome for the interpretable prescriptive policy is determined, where the difference quantifies a trade-off between the first policy and interpretability of the interpretable prescriptive policy in terms of expected outcome. These optional features contribute to the advantage of providing guidance to a decision-maker.

In some embodiments, the prescriptive tree is adjusted based on one or more pre-determined constraints, and a difference between the best expected outcome and an expected outcome for an interpretable prescriptive policy. These optional features contribute to the advantage of finetuning the size/depth of the prescriptive tree, such that the segmentation the prescriptive tree is trained for results in one or more rules appropriate for an application use.

These and other aspects, features and advantages of embodiments of the invention will be understood with reference to the drawing figures, and detailed description herein, and will be realized by means of the various elements and combinations particularly pointed out in the appended claims. It is to be understood that both the foregoing general description and the following brief description of the drawings and detailed description of embodiments of the invention are exemplary and explanatory of preferred embodiments of the invention, and are not restrictive of embodiments of the invention, as claimed.

### BRIEF DESCRIPTION OF THE DRAWINGS

The subject matter which is regarded as embodiments of the invention are particularly pointed out and distinctly claimed in the claims at the conclusion of the specification. The foregoing and other objects, features, and advantages of embodiments of the invention are apparent from the following detailed description taken in conjunction with the accompanying drawings in which:

FIG. 1 illustrates an example computing architecture for implementing integrated segmentation and interpretable prescriptive polices generation, in accordance with an embodiment of the invention;

FIG. 2 illustrates an example segmentation and policies generation system, in accordance with an embodiment of the invention;

FIG. 3 illustrates an example prescriptive tree, in accordance with an embodiment of the invention;

FIG. 4 is a flowchart for an example process for integrated segmentation and prescriptive policies generation, in accordance with an embodiment of the invention;

FIG. 5 depicts a cloud computing environment according to an embodiment of the present invention;

FIG. 6 depicts abstraction model layers according to an embodiment of the present invention; and

FIG. 7 is a high level block diagram showing an information processing system useful for implementing an embodiment of the invention.

The detailed description explains the preferred embodiments of the invention, together with advantages and features, by way of example with reference to the drawings.

### DETAILED DESCRIPTION

Embodiments of the invention generally relate to artificial intelligence (AI), and more specifically, to a method and system for integrated segmentation and prescriptive policies generation. One embodiment of the invention provides a method for integrated segmentation and prescriptive policies generation. The method comprises training a first AI model and a second model based on training data. The first AI model comprises a teacher model (e.g., a highly complex black box model, such as a neural network) trained to determine a likelihood of a desired outcome (e.g., purchase an item) for a given action. The second model comprises a prescriptive tree trained for segmentation (i.e., constructs a decision tree with a customized/user-defined splitting criterion (e.g., expected revenue maximization) which optimizes the desired outcome). The method further comprises determining, via the teacher model, a first policy that produces an optimal action. The optimal action provides a best expected outcome (although the optimal action may not be interpretable, e.g., the first policy involves fully personalized pricing produced by a black box model). The method further comprises applying, via the second model, a recursive segmentation algorithm to generate one or more interpretable prescriptive policies. Each interpretable prescriptive policy is less complex and more interpretable than the first policy, from the teacher model, that produces the optimal action. The method further comprises, for each interpretable prescriptive policy, determining, via the teacher model, an expected outcome for the interpretable prescriptive policy.

Another embodiment of the invention provides a system for integrated segmentation and prescriptive policies generation. The system comprises at least one processor, and a non-transitory processor-readable memory device storing instructions that when executed by the at least one processor causes the at least one processor to perform operations. The operations include training a first AI model and a second model based on training data. The first AI model comprises a teacher model (e.g., a highly complex black box model, such as a neural network) trained to determine a likelihood of a desired outcome (e.g., purchase an item) for a given action. The second model comprises a prescriptive tree trained for segmentation (i.e., constructs a decision tree with a customized/user-defined splitting criterion (e.g., expected revenue maximization) which optimizes the desired outcome). The operations further comprise determining, via the teacher model, a first policy that produces an optimal action. The optimal action provides a best expected outcome (although the optimal action may not be interpretable, e.g., the first policy involves fully personalized pricing produced by a black box model). The operations further comprise applying, via the second model, a recursive segmentation algorithm to generate one or more interpretable prescriptive policies. Each interpretable prescriptive policy is less complex and more interpretable than the first policy, from the teacher model, that produces the optimal action. The operations further comprise, for each interpretable prescriptive policy, determining, via the teacher model, an expected outcome for the interpretable prescriptive policy.

One embodiment of the invention provides a computer program product for integrated segmentation and prescriptive policies generation. The computer program product comprises a computer readable storage medium having program instructions embodied therewith, the program instructions executable by a processor to cause the processor to training a first AI model and a second model based on training data. The first AI model comprises a teacher model (e.g., a highly complex black box model, such as a neural network) trained to determine a likelihood of a desired outcome (e.g., purchase an item) for a given action. The second model comprises a prescriptive tree trained for segmentation (i.e., constructs a decision tree with a customized/user-defined splitting criterion (e.g., expected revenue maximization) which optimizes the desired outcome). The program instructions are further executable by the processor to cause the processor to determine, via the teacher model, a first policy that produces an optimal action. The optimal action provides a best expected outcome (although the optimal action may not be interpretable, e.g., the first policy involves fully personalized pricing produced by a black box model). The program instructions are further executable by the processor to cause the processor to apply, via the second model, a recursive segmentation algorithm to generate one or more interpretable prescriptive policies. Each interpretable prescriptive policy is less complex and more interpretable than the first policy, from the teacher model, that produces the optimal action. The program instructions are further executable by the processor to cause the processor to, for each interpretable prescriptive policy, determine, via the teacher model, an expected outcome for the interpretable prescriptive policy.

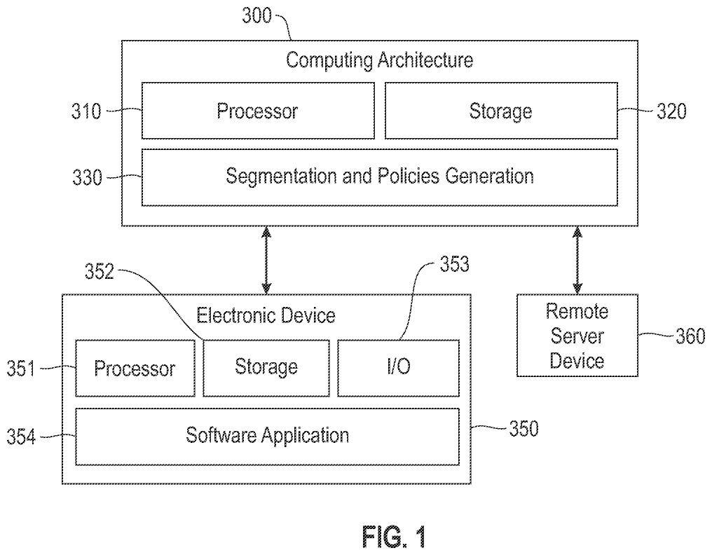

FIG. 1 illustrates an example computing architecture 300 for implementing integrated segmentation and interpretable prescriptive polices generation, in accordance with an embodiment of the invention. In one embodiment, the computing architecture 300 is a centralized computing architecture. In another embodiment, the computing architecture 300 is a distributed computing architecture.

In one embodiment, the computing architecture 300 comprises computation resources such as, but not limited to, one or more processor units 310 and one or more storage units 320. One or more applications may execute/operate on the computing architecture 300 utilizing the computation resources of the computing architecture 300. In one embodiment, the applications on the computing architecture 300 include, but are not limited to, a segmentation and policies generation system 330. As described in detail later herein, the system 330 is configured for simultaneous integrated segmentation and interpretable prescriptive policies generation via a teacher model and a prescriptive tree.

In one embodiment, the system 330 is configured to exchange data with one or more electronic devices 350 and/or one or more remote server devices 360 over a connection (e.g., a wireless connection such as a Wi-Fi connection or a cellular data connection, a wired connection, or a combination of the two).

In one embodiment, an electronic device 350 comprises one or more computation resources such as, but not limited to, one or more processor units 351 and one or more storage units 352. One or more applications may execute/operate on an electronic device 350 utilizing the one or more computation resources of the electronic device 350 such as, but not limited to, one or more software applications 354 loaded onto or downloaded to the electronic device 350. Examples of software applications 354 include, but are not limited to, artificial intelligence (AI) applications, etc.

Examples of an electronic device 350 include, but are not limited to, a desktop computer, a mobile electronic device (e.g., a tablet, a smart phone, a laptop, etc.), a wearable device (e.g., a smart watch, etc.), an Internet of Things (IoT) device, etc.

In one embodiment, an electronic device 350 comprises one or more input/output (I/O) units 353 integrated in or coupled to the electronic device 350, such as a keyboard, a keypad, a touch interface, a display screen, etc. A user may utilize an I/O module 353 of an electronic device 350 to configure one or more user preferences, configure one or more parameters (e.g., constraints, etc.), provide input (e.g., selection), etc.

In one embodiment, an electronic device 350 and/or a remote server device 360 may be a source of at least one of the following: training data, or a trained model.

In one embodiment, the system 330 may be accessed or utilized by one or more online services (e.g., AI services) hosted on a remote server device 360 and/or one or more software applications 354 (e.g., AI applications) operating on an electronic device 350. For example, in one embodiment, a virtual assistant, a search engine, or another type of software application 354 operating on an electronic device 350 can invoke the system 330 to perform an AI task.

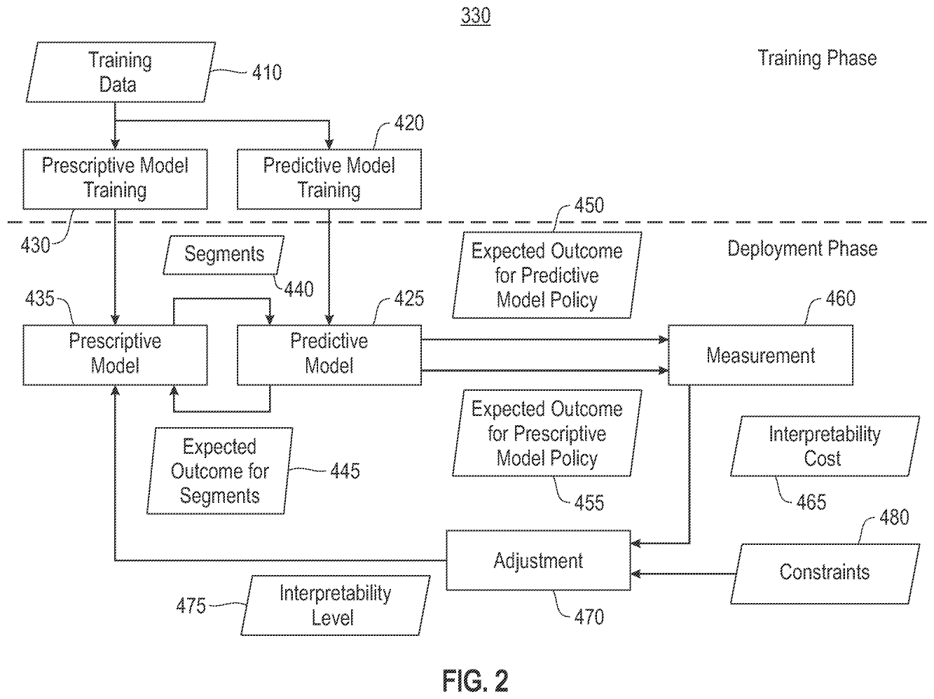

FIG. 2 illustrates an example segmentation and policies generation system 330, in accordance with an embodiment of the invention. In one embodiment, the system 330 has at least two different operating phases: a training phase during which one or more models are trained, and a deployment phase during which the one or more models are deployed for evaluation.

In one embodiment, the system 330 comprises a predictive model training unit 420. In the training phase, the predictive model training unit 420 is configured to: (1) receive, as input, training data 410, and (2) train an AI predictive model 425 for classification based on the training data 410. In one embodiment, the predictive model 425 comprises a non-parametric, teacher model. For example, in one embodiment, the teacher model is a highly complex black box machine learning model, such as a neural network. For expository purposes, the terms “predictive model” and “teacher model” are used interchangeably in this specification.

In one embodiment, the predictive model 425 is trained to evaluate (i.e., predict) a likelihood/probability of a desired outcome (i.e., successful outcome) for a given action (i.e., a success probability for a given action). For example, assume an application use (i.e., use case) involves pricing, an action is a particular price for an item or a product, and a successful outcome is a customer purchasing the item or the product at the particular price. In one embodiment, for the pricing, the predictive model 425 is utilized to determine success probability of the customer purchasing the item or the product at different prices.

In one embodiment, the system 330 comprises a prescriptive model training unit 430. In the training phase, the prescriptive model training unit 430 is configured to: (1) receive, as input, training data 410, and (2) train a prescriptive model 435 for segmentation based on the training data 410. In one embodiment, the prescriptive model 435 is trained using a specialized tree algorithm, resulting in a prescriptive tree including a root node and one or more leaf nodes. For expository purposes, the terms “prescriptive model” and “prescriptive tree” are used interchangeably in this specification.

In one embodiment, a path from the root node of the prescriptive tree to a particular leaf node of the tree specifies a particular segment of a population. In one embodiment, a leaf node of the prescriptive tree is prescribed a policy for a particular segment of a population specified by a path from the root node of the tree to the leaf node, wherein the policy is defined by a set of rules/items which produce the same action, and the rules/items have similar covariates. In one embodiment, a set of rules/items that have a similar optimal action, as evaluated by the predictive model 425, are selected to define a leaf node of the prescriptive tree.

In one embodiment, the prescriptive model 435 performs integrated segmentation which comprises constructing a decision tree with a customized/user-defined splitting criterion (e.g., expected revenue maximization) which optimizes a desired outcome for a given action. In one embodiment, the integrated segmentation performed is as follows: Each split of the prescriptive tree (e.g., on a feature of a product or a customer) separates data into two data sets. An estimated optimal action for each data set can be determined via the predictive model 425 (i.e., teacher model) which evaluates an expected outcome at each action, and chooses the optimal action. A split which results in the largest gain in estimated expected outcome is selected, and different actions are offered to resulting splits. The products are continuously recursively split into data sets, and the recursive splitting terminates once the tree reaches a given depth. Each leaf node represents a segment which will be assigned the same action.

In one embodiment, the training data 410 comprises historical data. In one embodiment, the training data 410 used to train both the predictive model 425 and the prescriptive model 435 is the same.

In one embodiment, in the deployment phase, the system 330 utilizes the predictive model 425 for evaluation. In one embodiment, the evaluation includes the predictive model 425 generating a policy (i.e., predictive model policy) that produces an optimal action. The optimal action provides a best expected (i.e., potential) outcome (i.e., the highest success probability). A policy that produces an optimal action, however, is likely complex and may not be interpretable by a decision-maker (i.e., a complex policy, e.g., a policy involving fully personalized pricing produced by a black box model). For expository purposes, the terms “complex policy”, “predictive model policy”, and “predictive policy” are used interchangeably in this specification.

In one embodiment, in the deployment phase, the system 330 utilizes the prescriptive model 435 for interpretable prescriptive policies generation. For example, in one embodiment, in the deployment phase, the system 330 feeds a complex policy that produces an optimal action and is generated by the predictive model 425 to the prescriptive model 435, and the prescriptive model 435 distills the complex policy into a simple policy that is interpretable by a decision-maker. In one embodiment, in the deployment phase, the prescriptive model 435 is configured to: (1) receive a complex policy (e.g., from the predictive model 425), and (2) apply a customized recursive partitioning/segmentation algorithm to generate one or more simple policies (i.e., prescriptive model policies), wherein each simple policy is less complex and more interpretable by a decision-maker than the complex policy, from the predictive model 425, that produces the optimal action (i.e., the simple policies are interpretable prescriptive policies). For expository purposes, the terms “simple policy”, “prescriptive model policy”, and “prescriptive policy” are used interchangeably in this specification.

In one embodiment, a simple policy generated by the prescriptive model 435 includes a set of actions/rules that define a particular leaf node of the prescriptive tree and that correspond to a particular segment 440 of a population. Each segment 440 produces a particular expected outcome (i.e., action), as evaluated by the predictive model 425.

In one embodiment, in the deployment phase, the system 330 feeds each simple policy generated by the prescriptive model 435 to the predictive model 425 for evaluation. In one embodiment, the predictive model 425 is configured to: (1) receive one or more segments 440 (e.g., from the prescriptive model 435), wherein each segment 440 represents a simple policy, and (2) for each segment 440, determine an expected outcome 445 for the segment 440, wherein the expected outcome 445 comprises a success probability for the segment 440. The predictive model 425 enables comparison of success probabilities for different segments 440.

For example, for an application use involving pricing, assume an expected outcome is expected revenue. In one embodiment, for the pricing, the predictive model 425 is utilized to determine expected revenue for different prices of an item or a product.

In one embodiment, the predictive model 425 and the prescriptive model 435 are intelligent agents that interact with each other. The prescriptive model 435 is a student model (i.e., corresponds to the learner in machine learning algorithms), and the predictive model 425 is a teacher model (i.e., which determines the loss function to facilitate the finetuning/adjusting/updating of the prescriptive model 435).

In one embodiment, the system 330 comprises a measurement unit 460. In one embodiment, in the deployment phase, the measurement unit 460 is configured to: (1) receive a first expected outcome 450 (e.g., from the predictive model 425), wherein the first expected outcome 450 is an evaluation of a complex policy generated by the predictive model 425, (2) receive a second expected outcome 455 (e.g., from the predictive model 425), wherein the second expected outcome 455 is an evaluation of a simple policy generated by the prescriptive model 435, and (3) measure a cost 465 of interpretability (“interpretability cost”) based on the first expected outcome 450 and the second expected outcome 455, wherein the interpretability cost 465 represents a difference between expected outcomes for the complex policy and the simple policy. In one embodiment, the interpretability cost 465 is a measurement quantifying a trade-off between a complex policy and interpretability of a simple policy in terms of expected outcome. In one embodiment, the interpretability cost 465 represents how far (i.e., distance) an expected outcome for a simple policy is from an optimal action (i.e., an expected outcome for a complex policy). For example, if the interpretability cost 465 is significant (e.g., exceeds a pre-determined threshold/tolerance), a decision-maker may prefer a complex policy that provides more predictive accuracy over a simple policy that provides more interpretability. As another example, if the interpretability cost 465 is not significant (e.g., does not exceed the pre-determined threshold/tolerance), a decision-maker may prefer a simple policy that provides more interpretability over a complex policy that provides more predictive accuracy. In one embodiment, the interpretability cost is utilized as a loss function to facilitate the finetuning/adjusting/updating of the prescriptive model 435.

In one embodiment, complexity of the prescriptive model 435 is adjustable (i.e., customizable or updateable). In one embodiment, the system 330 comprises an adjustment unit 470. In one embodiment, in the deployment phase, the adjustment unit 470 is configured to: (1) receive an interpretability cost 465 (e.g., from the measurement unit 460), (2) receive one or more constraints 480 for a particular application use (e.g., a maximum number of rules, a pre-determined threshold/tolerance for interpretability cost), and (3) determine a level 475 of interpretability (“interpretability level”) suitable for the application use based on the interpretability cost 465 and/or the one or more constraints 480. The system 330 utilizes the interpretability level 475 to finetune/adjust/update the prescriptive model 435 in terms of the size/depth of the prescriptive tree, such that the segmentation performed by prescriptive model 435 results in one or more rules appropriate for the application use.

In one embodiment, the system 330 is deployed for different application uses such as, but not limited to, targeted pricing (e.g., grocery item price optimization, airline seat upgrade pricing), targeted promotion (e.g., grocery item discount optimization), healthcare (e.g., personalized/precision medicine), customer relationship management (CRM), etc.

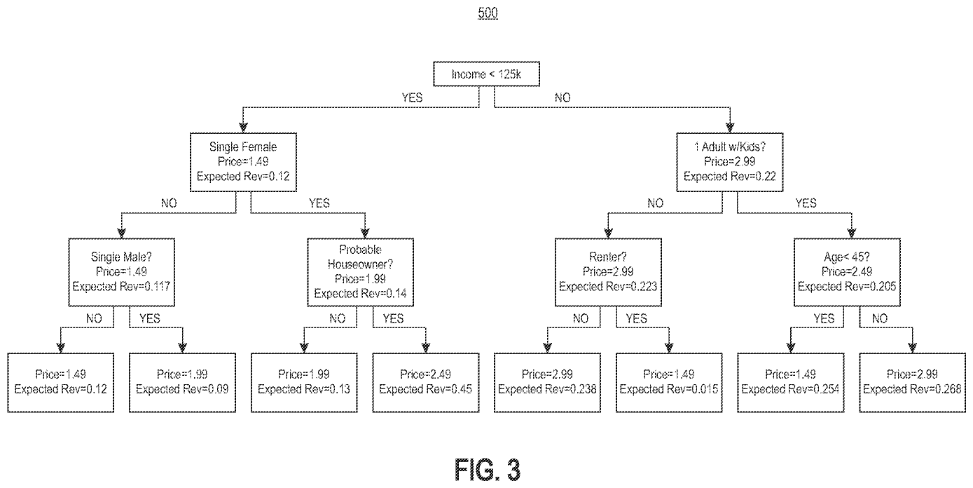

FIG. 3 illustrates an example prescriptive tree 500, in accordance with an embodiment of the invention. In one embodiment, the prescriptive tree 500 is deployed as a prescriptive model 435 for pricing. Each leaf node of the tree 500 corresponds to a segment of customers (i.e., the segment has particular demographics such as income, age, gender, family situation, living situation, etc.), is prescribed a pricing policy that defines a particular price for a product (e.g., a grocery item), and includes an expected revenue for the pricing policy (as evaluated by a teacher model, such as the predictive model 425 in FIG. 2). Each leaf node represents an interpretable personalized pricing policy for a particular segment of customers.

Table 1 below provides an example process for training and deploying a teacher model and a prescriptive tree for targeted pricing, in accordance with an embodiment of the invention.

TABLE 1
Notations: xᵢ ∈ R^{d} are features which describe the i^{th} item, pⁱ ∈ R is the
price assigned to the item, and yᵢ ∈ {0, 1} is whether the item sold (1)
or not (0)
Train a predictive teacher model by solving an empirical risk minimization
problem f* = arg min_{f∈F} Σᵢⁿ L(xᵢ, pᵢ, yᵢ; f), which gives an estimate of the
conditional probability of a sale f*(x, p) = \hat{P}(y|x, p).
For a surrogate model, define the revenue maximization criterion, R(Sₗ) =
maxₚ Σ_{i∈S}_{ₗ} pf*(xᵢ, p), where Sₗ is the subset of observations which belong
to leaf l of a decision tree. The goal of the surrogate learning algorithm is
to segment the data into L leaves, S₁, S₂, . . . , S_{L} such that the total sum
of predicted revenues is maximized
To accomplish this, use a heuristic called recursive partitioning, i.e.,
consider a decision split S₁(j, s) = {i ∈ [n]|x_{i,j} ≤ s} and S₂(j, s) =
{i ∈ [n]|x_{i,j} ≤ s}.
Optimize over j and s to find the best split of the tree: max_{j,s} R(S₁(j, s)) +
R(S₂(j, s))

In one embodiment, shallow prescriptive trees with fewer segments which translate into fewer pricing policies are desirable. Incorporating a teacher model controls for observed confounding variables at any depth, rather than assuming they are the same, therefore ensuring that confounding effects are minimized.

In one embodiment, a prescriptive tree and a teacher model are deployed for a healthcare setting involving personalized/precision medicine. Both models are trained based on publicly available patient datasets (e.g., Consortium 2009) which contain true patient-specific optimal doses of a particular medicine, and also include patient-level covariates such as clinical factors, demographic variables, and genetic information. For this particular application use, a successful outcome represents when a correct dosage is given. The system 330 is configured to train a teacher model based on the patient datasets, resulting in a trained teacher model that predicts success probability of a dosage given a patient's covariates. The system 330 is configured to train a prescriptive tree based on the same patient datasets. An optimal dosage that maximizes the success rate is determined via the prescriptive tree.

In one embodiment, a prescriptive tree and a teacher model are deployed for a different healthcare setting involving treatment for patients with cancer/chronic diseases. For this particular application use, a successful outcome represents one of the following: a 5-year survival rate for patients with cancer/chronic diseases, a recovery rate from a certain disease, a patient not returning to the ER within a certain time frame, or a patient not having certain side effects. An objective can be to maximize the success probability (i.e., success rate, e.g., survival rate) given patient covariates, by optimizing the treatment.

In one embodiment, a prescriptive tree and a teacher model are deployed for a CRM setting. For this particular application use, a successful outcome represents a customer is who is satisfied after a solution has been provided to address a complaint. An objective is to choose a most cost-effective solution from different compensation strategies with respect to the severity of a complaint.

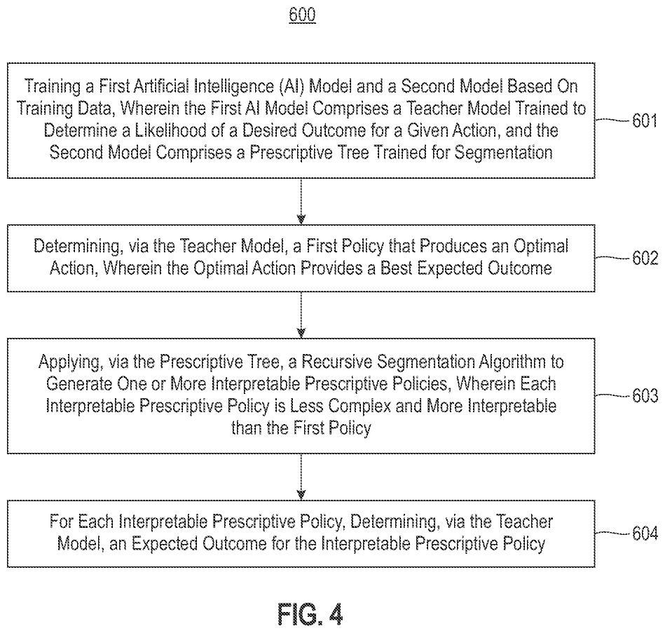

FIG. 4 is a flowchart for an example process 600 for integrated segmentation and prescriptive policies generation, in accordance with an embodiment of the invention. Process block 601 includes training a first AI model (e.g., predictive model 425 in FIG. 2) and a second model (e.g., prescriptive model 435 in FIG. 2) based on training data (e.g., training data 410 in FIG. 2), wherein the first model comprises a prescriptive teacher model trained to determine a likelihood of a desired outcome for a given action, and the second model comprises a prescriptive tree trained for segmentation. Process block 602 includes determining, via the teacher model, a first policy that produces an optimal action, wherein the optimal action provides a best expected outcome (e.g., expected outcome 450 in FIG. 2). Process block 603 includes applying, via the prescriptive tree, a recursive segmentation algorithm to generate one or more interpretable prescriptive policies (e.g., segments 440 in FIG. 2), wherein each interpretable prescriptive policy is less complex and more interpretable than the first policy. Process block 604 includes, for each interpretable prescriptive policy, determining, via the first model, an expected outcome for the interpretable prescriptive policy (e.g., expected outcomes 445 in FIG. 2).

In one embodiment, process blocks 601-604 are performed by one or more components of the system 330.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. In one embodiment, this cloud model includes at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and personal digital assistants).

Resource pooling: the provider's computing resources are pooled to serve multiple consumers using a multi-tenant model, with different physical and virtual resources dynamically assigned and reassigned according to demand. In one embodiment, there is a sense of location independence in that the consumer generally has no control or knowledge over the exact location of the provided resources but is able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. In one embodiment, it is managed by the organization or a third party and exists on-premises or off-premises.

Community cloud: the cloud infrastructure is shared by several organizations and supports a specific community that has shared concerns (e.g., mission, security requirements, policy, and compliance considerations). In one embodiment, it is managed by the organizations or a third party and exists on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load balancing between clouds).

A cloud computing environment is service oriented with a focus on statelessness, low coupling, modularity, and semantic interoperability. At the heart of cloud computing is an infrastructure that includes a network of interconnected nodes.

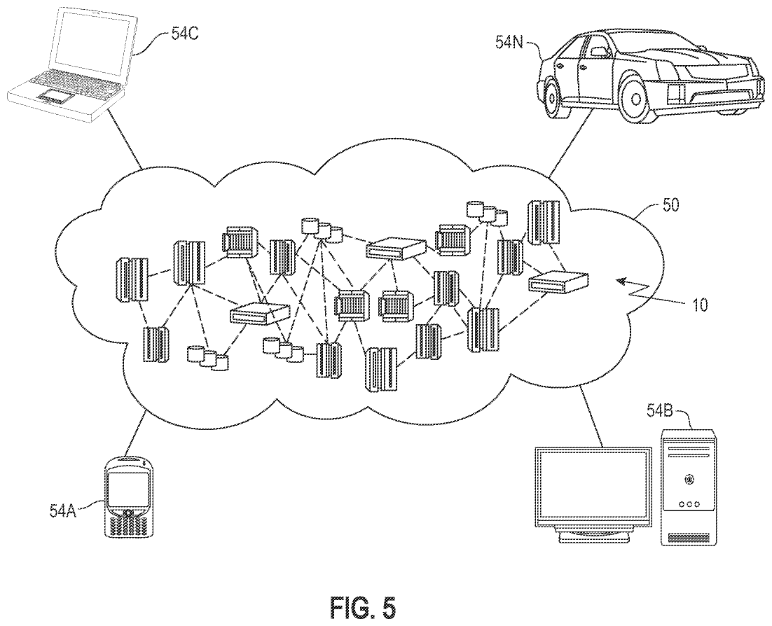

FIG. 5 depicts a cloud computing environment 50 according to an embodiment of the present invention. As shown, in one embodiment, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N communicate. In one embodiment, nodes 10 communicate with one another. In one embodiment, they are grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 10 and cloud computing environment 50 can communicate with any type of computerized device over any type of network and/or network addressable connection (e.g., using a web browser).

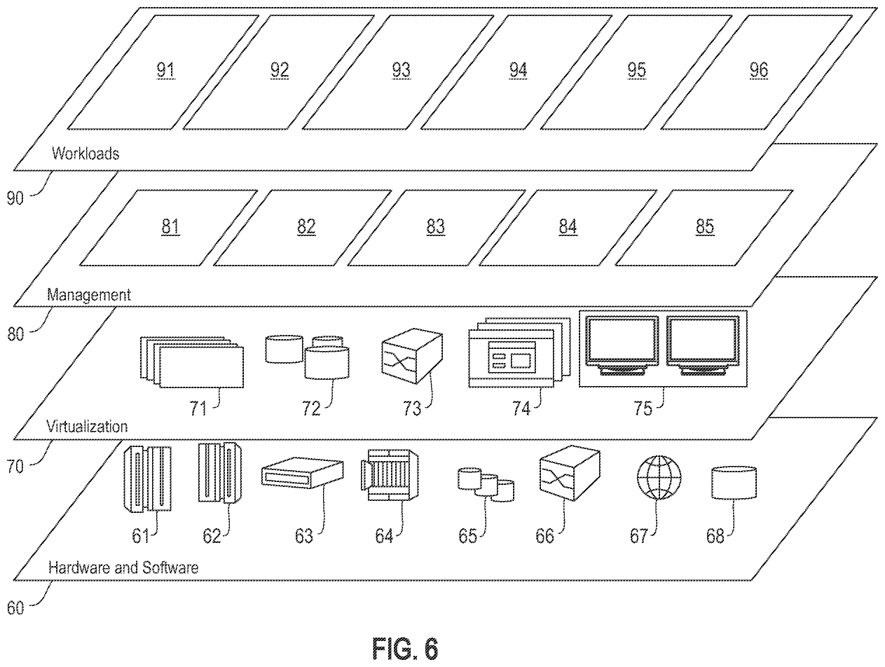

FIG. 6 depicts a set of functional abstraction layers provided by cloud computing environment 50 according to an embodiment of the present invention. It should be understood in advance that the components, layers, and functions shown in FIG. 6 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

In one embodiment, virtualization layer 70 provides an abstraction layer from which the following examples of virtual entities are provided: virtual servers 71; virtual storage 72; virtual networks 73, including virtual private networks; virtual applications and operating systems 74; and virtual clients 75.

In one embodiment, management layer 80 provides the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one embodiment, these resources include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

In one embodiment, workloads layer 90 provides examples of functionality for which the cloud computing environment is utilized. In one embodiment, examples of workloads and functions which are provided from this layer include: mapping and navigation 91; software development and lifecycle management 92; virtual classroom education delivery 93; data analytics processing 94; transaction processing 95; and AI 96 (e.g., a segmentation and policies generation system 330 (FIG. 1)).

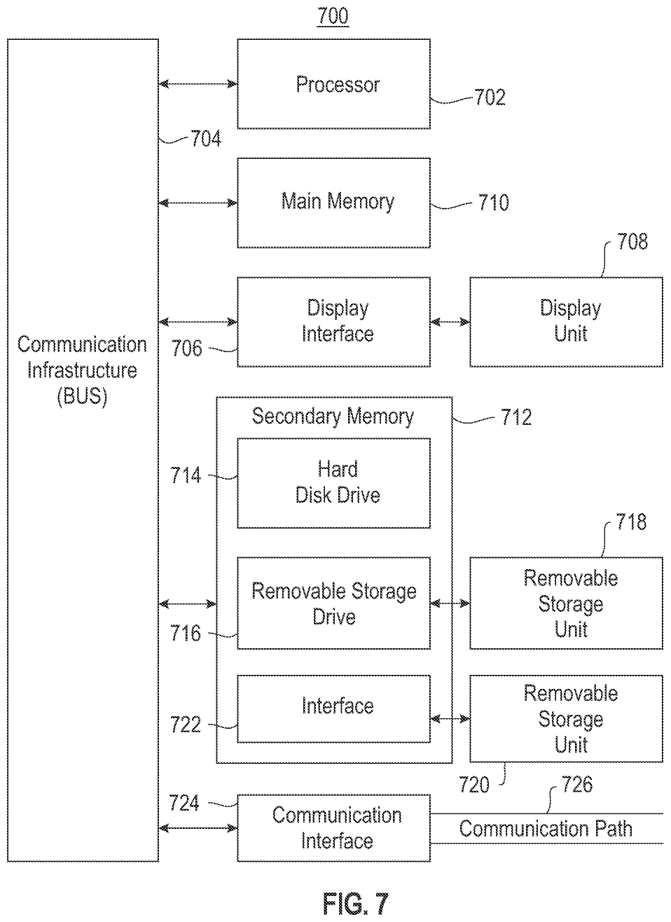

FIG. 7 is a high level block diagram showing an information processing system 700 useful for implementing one embodiment of the invention. The computer system includes one or more processors, such as processor 702. The processor 702 is connected to a communication infrastructure 704 (e.g., a communications bus, cross-over bar, or network).

The computer system can include a display interface 706 that forwards graphics, text, and other data from the voice communication infrastructure 704 (or from a frame buffer not shown) for display on a display unit 708. In one embodiment, the computer system also includes a main memory 710, preferably random access memory (RAM), and also includes a secondary memory 712. In one embodiment, the secondary memory 712 includes, for example, a hard disk drive 714 and/or a removable storage drive 716, representing, for example, a floppy disk drive, a magnetic tape drive, or an optical disk drive. The removable storage drive 716 reads from and/or writes to a removable storage unit 718 in a manner well known to those having ordinary skill in the art. Removable storage unit 718 represents, for example, a floppy disk, a compact disc, a magnetic tape, or an optical disk, etc. which is read by and written to by removable storage drive 716. As will be appreciated, the removable storage unit 718 includes a computer readable medium having stored therein computer software and/or data.

In alternative embodiments, the secondary memory 712 includes other similar means for allowing computer programs or other instructions to be loaded into the computer system. Such means include, for example, a removable storage unit 720 and an interface 722. Examples of such means include a program package and package interface (such as that found in video game devices), a removable memory chip (such as an EPROM, or PROM) and associated socket, and other removable storage units 720 and interfaces 722, which allows software and data to be transferred from the removable storage unit 720 to the computer system.

In one embodiment, the computer system also includes a communication interface 724. Communication interface 724 allows software and data to be transferred between the computer system and external devices. In one embodiment, examples of communication interface 724 include a modem, a network interface (such as an Ethernet card), a communication port, or a PCMCIA slot and card, etc. In one embodiment, software and data transferred via communication interface 724 are in the form of signals which are, for example, electronic, electromagnetic, optical, or other signals capable of being received by communication interface 724. These signals are provided to communication interface 724 via a communication path (i.e., channel) 726. In one embodiment, this communication path 726 carries signals and is implemented using wire or cable, fiber optics, a phone line, a cellular phone link, an RF link, and/or other communication channels.

Embodiments of the invention may be a system, a method, and/or a computer program product at any possible technical detail level of integration. The computer program product may include a computer readable storage medium (or media) having computer readable program instructions thereon for causing a processor to carry out aspects of embodiments of the invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of embodiments of the invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of embodiments of the invention.

Aspects of embodiments of the invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

From the above description, it can be seen that embodiments of the invention provide a system, computer program product, and method for implementing the embodiments of the invention. Embodiments of the invention further provide a non-transitory computer-useable storage medium for implementing the embodiments of the invention. The non-transitory computer-useable storage medium has a computer-readable program, wherein the program upon being processed on a computer causes the computer to implement the steps of embodiments of the invention described herein. References in the claims to an element in the singular is not intended to mean “one and only” unless explicitly so stated, but rather “one or more.” All structural and functional equivalents to the elements of the above-described exemplary embodiment that are currently known or later come to be known to those of ordinary skill in the art are intended to be encompassed by the present claims. No claim element herein is to be construed under the provisions of 35 U.S.C. section 112, sixth paragraph, unless the element is expressly recited using the phrase “means for” or “step for.”

The terminology used herein is for the purpose of describing particular embodiments of the invention only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed.

The descriptions of the various embodiments of the invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for integrated segmentation and prescriptive policies generation, comprising:
training a first artificial intelligence (AI) model and a second model based on training data, wherein the first AI model comprises a teacher model trained to determine a likelihood of a desired outcome for a given action, and the second model comprises a prescriptive tree trained for segmentation;
determining, via the teacher model, a first policy that produces an optimal action, wherein the optimal action provides a best expected outcome;
applying, via the prescriptive tree, a recursive segmentation algorithm to generate one or more interpretable prescriptive policies, wherein each interpretable prescriptive policy is less complex and more interpretable than the first policy; and
for each interpretable prescriptive policy, determining, via the teacher model, an expected outcome for the interpretable prescriptive policy.

2. The method of claim 1, wherein the segmentation the prescriptive tree is trained for comprises constructing a decision tree with a user-defined splitting criterion which optimizes the desired outcome.

3. The method of claim 1, wherein the teacher model is a neural network.

4. The method of claim 1, wherein each leaf of the prescriptive tree represents an interpretable prescriptive policy for a particular segment of a population, and demographics of the segment are specified by a path from a root of the prescriptive tree to the leaf node.

5. The method of claim 4, wherein each model is deployed for use in an application involving targeted pricing, each interpretable prescriptive policy represents an optimal product price for a segment of customers, and the best expected outcome represents a maximum expected revenue from the targeted pricing.

6. The method of claim 4, wherein each model is deployed for use in an application involving targeted promotion, each interpretable prescriptive policy represents an optimal product discount for a segment of customers, and the best expected outcome represents a maximum expected revenue from the targeted promotion.

7. The method of claim 4, wherein each model is deployed for use in an application involving personalized medicine, each interpretable prescriptive policy represents an optimal treatment for a segment of patients, and the best expected outcome represents a maximum success rate from the personalized medicine.

8. The method of claim 1, further comprising:
selecting from the one or more interpretable prescriptive policies based on each expected outcome for each interpretable prescriptive policy.

9. The method of claim 1, further comprising:
for each interpretable prescriptive policy, determining a difference between the best expected outcome and an expected outcome for the interpretable prescriptive policy, wherein the difference quantifies a trade-off between the first policy and interpretability of the interpretable prescriptive policy in terms of expected outcome.

10. The method of claim 9, further comprising:
adjusting the prescriptive tree based on one or more pre-determined constraints, and a difference between the best expected outcome and an expected outcome for an interpretable prescriptive policy.

11. A system for integrated segmentation and prescriptive policies generation, comprising:
at least one processor; and
a non-transitory processor-readable memory device storing instructions that when executed by the at least one processor causes the at least one processor to perform operations including:
training a first artificial intelligence (AI) model and a second model based on training data, wherein the first AI model comprises a teacher model trained to determine a likelihood of a desired outcome for a given action, and the second model comprises a prescriptive tree trained for segmentation;
determining, via the teacher model, a first policy that produces an optimal action, wherein the optimal action provides a best expected outcome;
applying, via the prescriptive tree, a recursive segmentation algorithm to generate one or more interpretable prescriptive policies, wherein each interpretable prescriptive policy is less complex and more interpretable than the first policy; and
for each interpretable prescriptive policy, determining, via the teacher model, an expected outcome for the interpretable prescriptive policy.

12. The system of claim 11, wherein each leaf of the prescriptive tree represents an interpretable prescriptive policy for a particular segment of a population, and demographics of the segment are specified by a path from a root of the prescriptive tree to the leaf node.

13. The system of claim 12, wherein each model is deployed for use in an application involving targeted pricing, each interpretable prescriptive policy represents an optimal product price for a segment of customers, and the best expected outcome represents a maximum expected revenue from the targeted pricing.

14. The system of claim 12, wherein each model is deployed for use in an application involving targeted promotion, each interpretable prescriptive policy represents an optimal product discount for a segment of customers, and the best expected outcome represents a maximum expected revenue from the targeted promotion.

15. The system of claim 12, wherein each model is deployed for use in an application involving personalized medicine, each interpretable prescriptive policy represents an optimal treatment for a segment of patients, and the best expected outcome represents a maximum success rate from the personalized medicine.

16. The system of claim 11, wherein the operations further comprise:
selecting from the one or more interpretable prescriptive policies based on each expected outcome for each interpretable prescriptive policy.

17. The system of claim 11, wherein the operations further comprise:
for each interpretable prescriptive policy, determining a difference between the best expected outcome and an expected outcome for the interpretable prescriptive policy, wherein the difference quantifies a trade-off between predictive accuracy of the first policy and interpretability of the interpretable prescriptive policy.

18. The system of claim 17, wherein the operations further comprise:
adjusting the prescriptive tree based on one or more pre-determined constraints, and a difference between the best expected outcome and an expected outcome for an interpretable prescriptive policy.

19. A computer program product for integrated segmentation and prescriptive policies generation, the computer program product comprising a computer readable storage medium having program instructions embodied therewith, the program instructions executable by a processor to cause the processor to:
train a first artificial intelligence (AI) model and a second model based on training data, wherein the first AI model comprises a teacher model trained to determine a likelihood of a desired outcome for a given action, and the second model comprises a prescriptive tree trained for segmentation;
determine, via the teacher model, a first policy that produces an optimal action, wherein the optimal action provides a best expected outcome;
apply, via the prescriptive tree, a recursive segmentation algorithm to generate one or more interpretable prescriptive policies, wherein each interpretable prescriptive policy is less complex and more interpretable than the first policy; and
for each interpretable prescriptive policy, determine, via the teacher model, an expected outcome for the interpretable prescriptive policy.

20. The computer program product of claim 19, wherein each leaf of the prescriptive tree represents an interpretable prescriptive policy for a particular segment of a population, and demographics of the segment are specified by a path from a root of the prescriptive tree to the leaf node.
